(19) United States
(12) Patent Application Publication
(10) Pub. No.: US 2008/0085300 A1
(43) Pub. Date: Apr. 10, 2008

(54) Hemostatic compositions and method of manufacture

(75) Inventors: Raymond J. Huey, Orange, CT (US); Denny Lo, Bethlehem, CT (US)

Correspondence Address: MICHAUD-DUFFY GROUP LLP, 306 INDUSTRIAL PARK ROAD, SUITE 206, MIDDLETOWN, CT, 06457, US

(73) Assignee: Z-Medica Corporation, Wallingford, CT (US)

(21) Appl. No.: 11/544,238

(22) Filed: Oct. 6, 2006

(51) Int. Cl. A61L 15/00

(52) U.S. Cl. 424/445

(57) ABSTRACT

A composition for promoting hemostasis is defined by a substrate in particle or pellet form and a hemostatic agent disposed on the substrate such that when using the composition to treat a bleeding wound, contacting the bleeding wound with the hemostatic agent causes blood to clot. The hemostatic agent may be a zeolite, bioactive glass, siliceous oxide, clay, biological hemostatic material, diatomaceous earth, or a combination of the foregoing. The substrate may be clay, glass, bioactive glass, diatomaceous earth, wax, polymer, plastic, metal, or a combination of the foregoing. A method of fabricating a hemostatic composition includes providing a substrate and a material having hemostatic characteristics. The hemostatic composition is fabricated by providing a material to operate as a carrier for the hemostatic material. The material is formed into particles or pellets. The material having hemostatic properties is applied or coated onto the particles of the carrier material.

### TECHNICAL FIELD

The present invention relates generally to hemostatic compositions and, more particularly, to compositions for use in controlling bleeding and their methods of manufacture.

### BACKGROUND OF THE INVENTION

Blood is a liquid tissue that includes red cells, white cells, corpuscles, and platelets dispersed in a liquid phase. The liquid phase is plasma, which includes acids, lipids, solubilzed electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of circumstances, animals, including humans, can be wounded. Often bleeding is associated with such wounds. In some circumstances, the wound and the bleeding are minor, and normal blood clotting functions in addition to the application of simple first aid are all that is required. Unfortunately, however, in other circumstances substantial bleeding can occur. These situations usually require specialized equipment and materials as well as personnel trained to administer appropriate aid. If such aid is not readily available, excessive blood loss can occur. When bleeding is severe, sometimes the immediate availability of equipment and trained personnel is still insufficient to stanch the flow of blood in a timely manner.

Moreover, severe wounds can often be inflicted in remote areas or in situations, such as on a battlefield, where adequate medical assistance is not immediately available. In these instances, it is important to stop bleeding, even in less severe wounds, long enough to allow the injured person or animal to receive medical attention.

In an effort to address the above-described problems, materials have been developed for controlling excessive bleeding in situations where conventional aid is unavailable or less than optimally effective. Although these materials have been shown to be somewhat successful, they are not effective enough for traumatic wounds and tend to be expensive. Furthermore, these materials are sometimes ineffective in all situations and can be difficult to apply as well as remove from a wound.

Additionally, or alternatively, the previously developed materials can produce undesirable side effects, particularly in instances in which they are misapplied to wounds or in which they are applied by untrained personnel. For example, because prior art blood clotting material is generally a powder or in fine particulate form, the surface area of the material is relatively large. The typical moisture content of a large surface area blood clotting material is generally up to about 15% of the total weight of the material. This combination of surface area and moisture content often produces an exothermic reaction upon the application of the material to blood. Depending upon the specific surface area and the specific amount of moisture, the resulting exothermia may be sufficient to cause discomfort to or even burn the patient. Although some prior art patents specifically recite the resulting exothermia as being a desirable feature that can provide cauterization of the wound, there exists the possibility that the tissue at and around the wound site can be undesirably damaged.

Based on the foregoing, it is a general object of the present invention to provide a hemostatic agent that overcomes or improves upon the problems and drawbacks associated with the prior art.

### SUMMARY OF THE INVENTION

According to one aspect of the present invention, a composition for promoting hemostasis is defined by a substrate in particle or pellet form and a hemostatic agent disposed on the substrate such that when using the composition to treat a bleeding wound, contacting the bleeding wound with the hemostatic agent causes the blood to clot. The hemostatic agent may be a zeolite, a bioactive glass, a siliceous oxide, a clay, a biological hemostatic material, diatomaceous earth, or the like or a combination of the foregoing, and the substrate may be a clay, a glass, a bioactive glass, diatomaceous earth, wax, polymer, plastic metal, or the like or a combination of the foregoing.

According to another aspect of the present invention, a method of fabricating a hemostatic composition includes providing a substrate and a material having hemostatic characteristics. The material having hemostatic characteristics is applied to the substrate. In this manner, the substrate functions as a vehicle for carrying the hemostatic material for delivery to a bleeding wound.

According to another aspect of the present invention, the hemostatic composition is fabricated by providing a material to operate as a carrier for the hemostatic material. The material is formed into particles or pellets. The material having hemostatic properties is applied or coated onto the particles of the carrier material.

An advantage of the present invention is that the hemostatic agent in combination with the carrier (particularly when the carrier is clay) reacts less exothermically with blood than if the hemostatic agent was used alone. This can be attributed at least in part to the fact that the surface area of the hemostatic agent exposed to the blood is reduced as compared with the use of hemostatic agent alone, and thereby moisture is drawn from the blood less aggressively. This tempers the exothermic effects experienced at the wound site. It is theorized that the less aggressive drawing of moisture from the blood is the result of a less rapid transfer of moisture into the substrate (particularly when the substrate is clay). However, the porous nature of the hemostatic agent still allows water to be wicked away to cause thickening of the blood, thereby facilitating the formation of clots.

Still another advantage of the present invention is that it is easily applied to an open wound. Particularly when the composition is in the form of particles, pellets, beads, rods, or granules, it can be readily removed from a sterilized packaging and deposited directly at the points from which blood emanates to cause clotting.

### BRIEF DESCRIPTION OF THE DRAWINGS

FIG. 1 is a schematic representation of a hemostatic agent of the present invention.

FIG. 2 is a schematic representation of a hemostatic agent of the present invention utilizing an adhesive to retain a material having hemostatic characteristics on a substrate.

### DETAILED DESCRIPTION OF THE PREFERRED EMBODIMENTS

Disclosed herein are compositions applicable to bleeding wounds to promote hemostasis and methods of manufacturing such compositions. These compositions generally comprise hemostatic agents as active ingredients that can minimize or stop blood flow by absorbing at least portions of the liquid phases of the blood, thereby promoting clotting.

In one embodiment of the present invention, the hemostatic agent is a zeolite or other molecular sieve material. The present invention is not limited in this regard, however, as other materials are also within the scope of the present invention. As used herein, the term “zeolite” refers to a crystalline form of aluminosilicate having the ability to be dehydrated without experiencing significant changes in the crystalline structure. The zeolite may include one or more ionic species such as, for example, calcium and sodium moieties. Typically, the zeolite is a friable material that is about 90% by weight calcium and about 10% by weight sodium. The calcium portion contains crystals that are about 5 angstroms in size, and the sodium portion contains crystals that are about 4 angstroms in size. The preferred molecular structure of the zeolite is an “A-type” crystal, namely, one having a cubic crystalline structure that defines round or substantially round openings.

The zeolite may be mixed with or otherwise used in conjunction with other materials having the ability to be dehydrated without significant changes in crystalline structure. Such materials include, but are not limited to, magnesium sulfate, sodium metaphosphate, calcium chloride, dextrin, combinations of the foregoing materials, and hydrates of the foregoing materials.

Zeolites for use in the disclosed applications may be naturally occurring or synthetically produced. Numerous varieties of naturally occurring zeolites are found as deposits in sedimentary environments as well as in other places. Naturally occurring zeolites that may be applicable to the compositions described herein include, but are not limited to, analcite, chabazite, heulandite, natrolite, stilbite, and thomosonite. Synthetically produced zeolites that may also find use in the compositions and methods described herein are generally produced by processes in which rare earth oxides are substituted by silicates, alumina, or alumina in combination with alkali or alkaline earth metal oxides.

Various materials may be mixed with, associated with, or incorporated into the zeolites to maintain an antiseptic environment at the wound site or to provide functions that are supplemental to the clotting functions of the zeolites. Exemplary materials that can be used include, but are not limited to, pharmaceutically-active compositions such as antibiotics, antifungal agents, antimicrobial agents, anti-inflammatory agents, analgesics, antihistamines (e.g., cimetidine, chloropheniramine maleate, diphenhydramine hydrochloride, and promethazine hydrochloride), compounds containing silver ions, and the like. Other materials that can be incorporated to provide additional hemostatic functions include ascorbic acid, tranexamic acid, rutin, and thrombin. Botanical agents having desirable effects on the wound site may also be added.

For application to an inert substrate or vehicle, the zeolite or other hemostatic agent is preferably in powder form. The powder form of the zeolite may be obtained by any suitable operation. For example, powdered zeolite may be obtained by grinding, crushing, rolling, or pulverizing coarser zeolite material. The present invention is not limited in this regard, however, as other methods of manipulating the zeolite into powder form known to those skilled in the art in which the present invention pertains may be employed.

In another embodiment of the present invention, the hemostatic agent coated onto the substrate is a bioactive glass. As used herein, the term “bioactive glass” refers to a surface-reactive glassy ceramic material that is biocompatible with human tissue. The composition of bioactive glass promotes a rapid ion exchange in aqueous environments. Bioactive glass can be defined by any one of a multitude of formulas, but it is predominantly a mixture of oxides. In general, bioactive glasses include silicon dioxide and calcium oxide. Other materials that may be incorporated into the bioactive glasses include, but are not limited to, sodium oxide and phosphorous pentoxide. Still other materials that may be added to the bioactive glasses include, but are not limited to, the pharmaceutically-active compositions described above.

In other embodiments, the material coated onto the substrate may be a siliceous oxide, a mixture of various siliceous oxides, any type of mesoporous material, a clay (e.g., attapulgite, bentonite, kaolin, or combinations thereof), diatomaceous earth, a biological composition having hemostatic characteristics (e.g., chitosan, thrombin, fibrin, Factor VII or similar enzymes, or compositions thereof), or any other composition having hemostatic properties. Such materials may be used in combination with zeolites or other molecular sieve materials.

Although the compositions and their methods of manufacture are described herein with reference to the active ingredient being a zeolite, it should be understood by those of skill in the art that the hemostatic agents and their methods of manufacture may additionally incorporate a bioactive glass, a siliceous oxide, a mesoporous material, a clay, diatomaceous earth, biological compositions, or any combination thereof to define the active ingredient.

In formulating the hemostatic agent, the zeolite is adhered to the substrate. The mechanism for adhesion between the zeolite and the substrate materials may be coulombic forces, a separate binding material, or an additional hemostatic agent. In embodiments in which a separate binding material is used, the material may be any biocompatible composition having sufficient properties that allow the composition to be retained on the substrate and to retain the active ingredient.

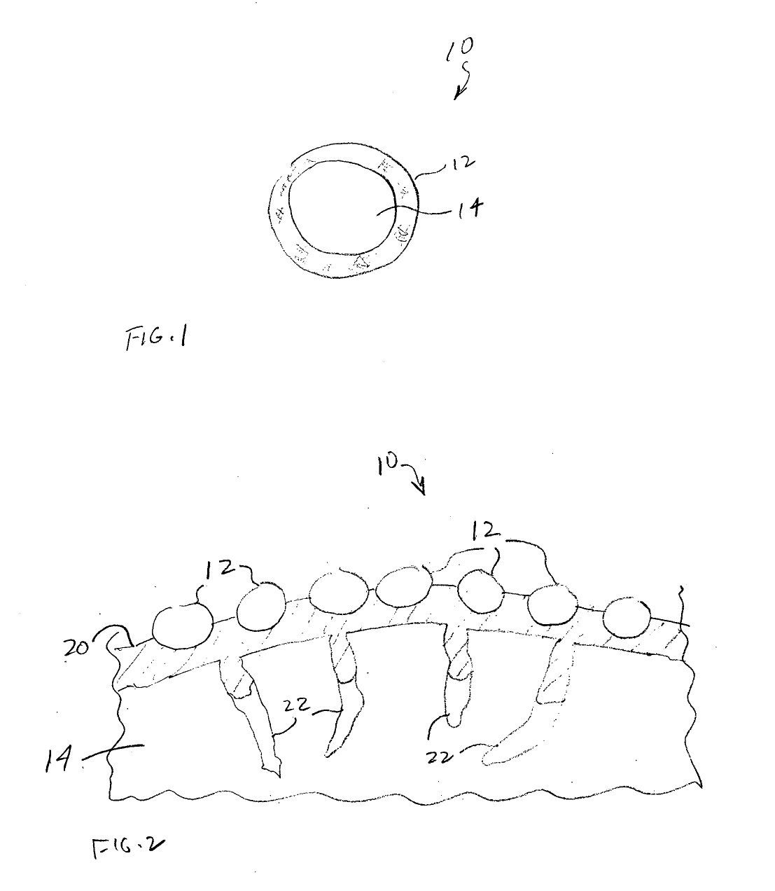

Referring now to FIG. 1, a hemostatic agent is shown generally at 10. In one exemplary embodiment, the hemostatic agent 10 comprises the zeolite, shown at 12, disposed on the substrate 14. The substrate 14 may be clay, an artificial or processed gel or gelling agent, or some other type of material such as a plastic that binds the zeolite 12 thereto or otherwise holds the zeolite.

In embodiments in which the substrate 14 is clay, any suitable clay may be used to form a clay core. One preferred type of clay is attapulgite clay, which is a crystalloid hydrated magnesium-aluminum silicate mineral. The crystalline structure of attapulgite clay causes it to include varying amounts of sodium, calcium, iron (in trivalent form), and aluminum, all of which are present in the forms of needles, fibers, and/or fibrous agglomerations. The adhesive qualities of attapulgite clay render it especially useful in retaining zeolites or other molecular sieve materials. The present invention is not limited in this regard, as other types of clays (such as bentonite, kaolin, and combinations thereof with or without attapulgite) can be used for the clay core. The present invention is also not limited to clays, as diatomaceous earth, waxes, polymers, glasses, metals, and combinations thereof with or without clay can be used for the substrate.

Artificial or specially processed colloidal gelling agents can also be used for the substrate 14. Such agents can be specifically tailored to bind zeolites or other molecular sieve materials by controlling the chemical compositions, rheologies, tribological aspects, and/or other properties thereof. One particular gelling agent is MIN-U-GEL® MB, which is available from Floridin of Quincy, Fla.

Although the substrate 14 is described hereinafter as being a clay material, it should be understood that any suitable material (e.g., artificial or specially processed colloidal gelling agents, plastics, bioactive glass, molecular sieve material) may be substituted for the clay.

In order to achieve a suitably homogenous mixture of the clay (or other substrate material), a relatively high shear is applied to the clay using a suitable mixing apparatus. Prior to shearing, the water content of the clay is measured and adjusted to be about 20% by weight to give a sufficiently workable mixture for extrusion and subsequent handling.

In embodiments in which the substrate 14 is a clay, the clay is particlized so as to be in the form of beads, pellets, granules, rods, spheres, or any other suitable morphology capable of functioning as a core structure. The clay may also be a mixture of various polymorphous shapes. The clay particles can be produced by any one of several methods. Such methods include mixing, extrusion, spheronizing, and the like. Equipment that can be utilized for the mixing, extruding, or spheronizing of the clay is available from Caleva Process Solutions Ltd. in Dorset, United Kingdom. Other methods include the use of a fluid bed or a pelletizing apparatus. Fluid beds for the production of clay particles are available from Glatt Air Technologies in Ramsey, N.J. Disk pelletizers for the production of clay particles are available from Feeco International, Inc., in Green Bay, Wis. The present invention is not limited in this regard, however, as other devices and methods for producing particlized clay are within the scope of the present invention.

The substrate 14 may be vitrified by firing the clay material to around 600 degrees C. Vitrification of the clay through repeated melting and cooling cycles allows the clay to be converted into a glassy substance. With increasing numbers of cycles, the crystalline structure is broken down to result in an amorphous composition. The amorphous nature of the clay allows it to maintain its structural integrity when subsequently wetted. As a result, the hemostatic agent 10 maintains its structural integrity when wetted during use, for example, when applied to blood. The present invention is not limited to substrates in which the clay is vitrified, however, as substrates formulated of clay that is not vitrified are within the scope of the present invention. In embodiments in which the clay of the substrate is not vitrified, the hemostatic agent 10 may be retained in a mesh bag or similar packaging for application to a bleeding wound.

The vitrified substrate 14 is coated with zeolite 12 using any one or a combination of various procedures. Pressure may be used to facilitate the coating of zeolite 12 onto the substrate 14, for example, by applying the zeolite to the clay core in a pressurized vessel. One procedure for coating the vitrified substrate 14 with zeolite 12 involves moistening the clay core and subsequently applying the zeolite in powder form. Moistening the substrate 14 may be effected by misting the clay core using any suitable misting apparatus.

Another procedure for coating the vitrified substrate 14 with zeolite 12 involves immersing the clay particles in a zeolite/clay slurry. Immersion of the clay particles in the zeolite/clay slurry may be accomplished by straining particles of the vitrified clay through the slurry using a mesh device or the like or by soaking the particles in the slurry. The present invention is not limited in this regard, as other methods of immersing the clay particles in the slurry are within the scope of the present invention. Because the clay of the substrate 14 has a natural affinity for the clay component of the zeolite/clay slurry, the adhesion of the zeolite 12 coated onto the substrate 14 after any final drying steps is improved.

Another procedure for coating the vitrified substrate 14 with zeolite involves spraying a zeolite/clay slurry onto the clay core. Any suitable apparatus capable of spraying zeolite/clay slurry can be used. One type of apparatus that can be used is a fluid bed apparatus. Fluid bed apparatuses are available from Glatt Air Technologies.

Once the vitrified substrate 14 is coated with zeolite 12, the zeolite is regenerated by driving out adsorbed water. Regeneration of the zeolite 12 is generally effected in purged dry air at a temperature of about 250 degrees C. to about 450 degrees C. In the alternative, the zeolite 12 can be regenerated in a vacuum oven to reduce the temperature as well as the duration of exposure of the zeolite to the heat.

In other embodiments, the substrate itself may be an active ingredient that is coated with another active ingredient. In one such embodiment, the substrate may be zeolite material onto which bioactive glass is coated. In another configuration, the substrate may be bioactive glass, and zeolite material may be coated thereon. The outer material may be coated onto the substrate using any suitable procedure, as described above.

Referring now to FIG. 2, biocompatible adhesives or the like may also be used to adhere the zeolite 12 (or other active ingredient) to the substrate 14. The hemostatic agent 10 is shown as including the substrate 14 and the zeolite 12 as well as an adhesive 20. In embodiments utilizing the adhesive 20, the adhesive is disposed on the substrate 14 such that portions of the adhesive anchor in pores 22 of the substrate. Given the tack qualities of the adhesive 20, zeolite 12 (or other active ingredient) is applied to and held in the adhesive. Preferably, the adhesive is sufficiently porous to facilitate the transfer of liquid from the zeolite 12 through adhesive and to the substrate 14.

### EXAMPLE 1

### Formulation of Zeolite-Coated Clay Particles

Clay having a moisture content of about 20% by weight was extruded through a die. The resulting clay pellets produced had diameters of 1.6 mm (millimeters) and lengths of one to two times the diameters. Dry 5A zeolite powder was applied to the moist clay pellets to produce a substantially uniform coating. The zeolite-coated pellets were heated to 300 degrees C. and maintained at that temperature for two hours to regenerate the zeolite. The pellets were then cooled to room temperature.

### EXAMPLE 2

### Comparison of Zeolite-Coated Clay Particles to Zeolite Pellets

The heat of adsorption of zeolite-coated clay particles (from Example 1) was compared to the heat of adsorption of comparably sized zeolite pellets. The zeolite-coated clay particles (25 g (grams)) were combined with distilled water (19 g). Both the clay particles and the distilled water were at room temperature before combining. Upon stirring, a peak temperature of 33 degrees C. was recorded. The same test using 5A zeolite particles produced a peak temperature of 79 degrees C. Thus, a given amount of zeolite-coated clay particles produced significantly less adsorption heat than the same amount of zeolite particles.

### EXAMPLE 3

### Formulations of Various Water/Zeolite/Clay Slurry for Spraying

Slurries of water/zeolite/clay were formulated for spraying onto clay substrates in the forms of pellets to give substantially uniform coatings with improved adhesion qualities. Clay was added to slurries of water and zeolite to improve the adhesion onto the clay substrates. The clay added to the slurries was the same clay that formed the clay substrates.

| Slurry | Water | | Zeolite | |
|---|---|---|---|---|
| No. | (g) | Clay (g) | (g) | |
| 1 | 50 | 5 | 5 | Dilute mixture; several sprays from |
| | | | | mister onto porous surface |
| | | | | yields little visible solid |
| | | | | residue. |
| 2 | 50 | 7.5 | 7.5 | Mixture can be sprayed and yields |
| | | | | suitable solid residue. |
| 3 | 50 | 10 | 10 | Viscous mixture; cannot be |
| | | | | sprayed. |

From the above slurries, it was determined that 50 g of water having 15 g of equal amounts of clay and zeolite (Slurry No. 2) provided a mixture that was suitable for spraying.

### EXAMPLE 4

### Assessment of Coating Adhesion

From the proportions of Slurry No. 2 (Example 3), additional slurries were formulated with varying amounts of zeolite-to-clay ratios. Pellets were formed by spraying each slurry onto a clay substrate, the clay of the substrate being matched to the clay of the slurry formulation.

After the pellets were heated to regenerate the zeolite, quantities (25 g) of the pellets were sifted with a 1680 micron mesh sieve. The sifting consisted of shaking the pellets on the sieve for about 10 seconds. The pellets were then reweighed.

| | | | | | Weight | Weight | |
|---|---|---|---|---|---|---|---|
| | | | | | Before | After | |
| Sample | Water | Clay | Zeolite | Zeolite/ | Sifting | Sifting | Weight |
| No. | (g) | (g) | (g) | Clay | (g) | (g) | Loss |
| A | 50 | 7.5 | 7.5 | 100% | 34.09 | 33.37 | 2.1% |
| B | 50 | 5 | 5 | 50% | 32.02 | 31.34 | 2.1% |
| C | 50 | 10 | 10 | 200% | 29.51 | 29.02 | 1.7% |
| D | 50 | 2.5 | 12.5 | 500% | 27.60 | 26.60 | 3.6% |

Accordingly, coating adhesion can be improved by incorporating some clay into the coating application via a spray process. This process can be scaled up and applied using fluid bed equipment capable of spraying a coating onto a moving bed of substrate material.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A composition for promoting hemostasis, said composition comprising:
a substrate in one of a particle and a pellet form;
a hemostatic agent coated on said substrate on substantially all exposed surfaces thereof; and
wherein upon contact with a bleeding wound said hemostatic agent causes blood flowing from said bleeding wound to clot.

2. The composition of claim 1, wherein said substrate is clay.

3. The composition of claim 2, wherein said clay is vitrified.

4. The composition of claim 2, wherein said clay is selected from the group consisting of attapulgite, bentonite, kaolin, and combinations of the foregoing.

5. The composition of claim 1, wherein said substrate is a colloidal gelling agent.

6. The composition of claim 1, wherein said substrate is a bioactive glass.

7. The composition of claim 1, wherein said substrate is a zeolite.

8. The composition of claim 1, wherein said substrate is diatomaceous earth.

9. The composition of claim 1, wherein said substrate is selected from the group consisting of waxes, polymers, glass, metals, and combinations of the foregoing.

10. The composition of claim 1, wherein said hemostatic agent is a zeolite.

11. The composition of claim 1, wherein said hemostatic agent is a bioactive glass.

12. The composition of claim 1, wherein said hemostatic agent is diatomaceous earth.

13. The composition of claim 1, wherein said hemostatic agent is selected from the group consisting of chitosan, thrombin, fibrin, Factor VII, and combinations of the foregoing.

14. The composition of claim 1, further comprising a binder to hold said hemostatic agent on said substrate.

15. The composition of claim 1, wherein said substrate is selected from the group consisting of clays, colloidal gelling agents, bioactive glasses, glasses, zeolites, waxes, polymers, metals, diatomaceous earth, and combinations of the foregoing, and
wherein said hemostatic agent is selected from the group consisting of clays, zeolites, bioactive glasses, diatomaceous earth, chitosan, thrombin, fibrin, Factor VII, and combinations of the foregoing.

16. A method of fabricating a hemostatic composition, said method comprising the steps of:
providing a substrate in one of a particle and a pellet form;
providing a material having hemostatic characteristics coated on said substrate on substantially all exposed surfaces thereof wherein upon contact with a bleeding wound said material having hemostatic characteristics causes blood flowing from said bleeding wound to clot; and
applying said material having hemostatic characteristics to said substrate.

17. The method of claim 16, wherein said step of applying said material having hemostatic characteristics to said substrate comprises immersing said substrate in a slurry of material that includes said material having hemostatic characteristics.

18. The method of claim 16, wherein said step of applying said material having hemostatic characteristics to said substrate comprises spraying said substrate with a slurry of material that includes said material having hemostatic characteristics.

19. The method of claim 16, further comprising drying said material having hemostatic characteristics to remove adsorbed moisture.

20. The method of claim 19, wherein said step of drying said material having hemostatic characteristics comprises heating said material to between about 250 degrees C. and about 450 degrees C.

21. The method of claim 16, wherein said substrate is a material selected from the group of materials consisting of clays, colloidal gelling agents, waxes, diatomaceous earth, polymers, glasses, metals, plastics, and combinations of the foregoing.

22. The method of claim 16, wherein said material having hemostatic characteristics is selected from the group consisting of molecular sieve materials, zeolites, bioactive glass materials, siliceous oxides, clays, diatomaceous earth, chitosan, thrombin, fibrin, Factor VII, and combinations of the foregoing.

23. The method of claim 22, wherein said clay is selected from the group consisting of attapulgite, bentonite, kaolin, and combinations of the foregoing.

24. A method of fabricating a hemostatic composition, said method comprising the steps of:
providing a clay material;
providing a material having hemostatic properties coated on said clay material on substantially all exposed surfaces thereof wherein upon contact with a bleeding wound said material having hemostatic properties causes blood flowing from said bleeding wound to clot;
forming said clay material into particles; and
coating said material having hemostatic properties onto said particles of said clay material.

25. The method of claim 24, wherein said step of forming said clay material into particles comprises extruding said clay material through a die.

26. The method of claim 24, wherein said step of forming said clay material into particles comprises spheronizing said clay material.

27. The method of claim 24, wherein said step of forming said clay material into particles comprises pelletizing said clay material.

28. The method of claim 24, further comprising drying said material having hemostatic properties.

29. The method of claim 24, further comprising vitrifying said clay material.

30. The method of claim 29, wherein said step of vitrifying said clay material comprises firing said clay particles to about 600 degrees C.

31. The method of claim 24, further comprising moistening said clay material prior to coating said material having hemostatic properties onto said particles of said clay material.

32. The method of claim 31, wherein said step of moistening said particles comprises misting said particles with water.

33. The method of claim 24, wherein said step of coating said material having hemostatic properties onto said particles of said clay material comprises immersing said particles of said clay material in a slurry of clay material and said material having hemostatic characteristics.

34. The method of claim 24, wherein said step of coating said material having hemostatic properties onto said particles of said clay material comprises spraying a slurry of clay material and material having hemostatic properties onto said particles of said clay material.

35. The method of claim 24, further comprising drying said material having hemostatic properties.

36. The method of claim 35, wherein said step of drying said material having hemostatic properties comprises heating said material to between about 250 degrees C. and about 450 degrees C.

37. The method of claim 35, wherein said step of drying said material having hemostatic properties comprises applying a vacuum to said material.

38. The method of claim 37, further comprising heating said material while under said vacuum.

39. The method of claim 24, wherein said clay material is selected from the group consisting of attapulgite, bentonite, kaolin, and combinations of the foregoing.

40. The method of claim 24, wherein said material having hemostatic characteristics is selected from the group consisting of molecular sieve materials, zeolites, bioactive glass materials, diatomaceous earth, clay, chitosan, thrombin, fibrin, Factor VII, siliceous oxides, and combinations of the foregoing.
